(19) United States
(12) Patent Application Publication
(10) Pub. No.: US 2026/0060886 A1
(43) Pub. Date: Mar. 5, 2026

(54) METHODS, SYSTEMS, AND DEVICES FOR TREATING ERECTILE DYSFUNCTION

(71) Applicants: Irwin Goldstein, San Diego, CA (US); Axel Erich Theuer, Kirchberg (DE); Nikolaus Hopfenzitz, Konstanz (DE); John F. Warlick, Talking Rock, GA (US)

(72) Inventors: Irwin Goldstein, San Diego, CA (US); Axel Erich Theuer, Kirchberg (DE); Nikolaus Hopfenzitz, Konstanz (DE); John F. Warlick, Talking Rock, GA (US)

(21) Appl. No.: 18/825,183

(22) Filed: Sep. 5, 2024

(51) Int. Cl. A61H 23/00; A61F 5/41

(52) U.S. Cl. CPC A61H 23/008; A61F 5/41; A61F 2005/412; A61H 2201/0111; A61H 2201/1409; A61H 2205/087

(57) ABSTRACT

A method for treating a penis of a patient comprises the steps of: positioning the penis within a penis-receiving space of a penis treatment device, causing the penis-receiving space to become at least partially filled with an energy wave propagating media, and emitting propagating energy waves into the penis-receiving space after causing the penis-receiving space to become at least partially filled with the energy wave propagating media whereby the penis within the penis-receiving body is subjected to the propagating energy waves. The penis-receiving space is at least partially encompassed by a layer of material having material characteristics causing the propagating energy waves that impinge upon the layer of material to be reflected therefrom. A step may be provided for causing the penis to become erect for enhancing exposure to and/or absorption of the propagating energy waves (e.g., via creating a negative pressure environment within the penis-receiving space).

### FIELD OF THE DISCLOSURE

The disclosures made herein relate generally to treatment of erectile dysfunction and, more particularly, to methods, systems, and devices for treating erectile dysfunction using propagating energy waves such as, for example, acoustic shock waves.

### BACKGROUND

Erectile Dysfunction (ED) is a medical condition in which a person is unable to achieve and/or sustain an erection suitable for achieving satisfactory sexual performance. A number of factors are believed to play a role in or be directly responsible for ED. Examples of these contributing factors include, but are not limited to, obesity, blood pressure, chronic illnesses such as diabetes, poor blood flow to the penis, smoking tobacco, alcoholism, and side-effects of other medications.

Treatments for ED currently include, for example, cessation of potential causes such as smoking tobacco and consumption of alcohol, hormone (testosterone) replacement, surgery, and administration of known-harmful pharmaceuticals (e.g., vardenafil, tadalafil, and sildenafil). Some of these pharmaceuticals are controversial for their incompatibility with nitrate drugs, and for their unwanted side-effects, such as effects on vision (blurring, loss of vision) and priapism. Vacuum enlargement pumps are another treatment technique for erectile dysfunction known in the art for many years. Such enlargement pumps aid in attaining and maintaining an erection through the use of negative pressure surrounding the penis for enabling increased blood flow into the penis. However, the results from use of a vacuum enlargement pump for treating erectile dysfunction generally offer only limited efficacy in regard to duration of erection and overall long-term outcome in regard to attaining and maintaining an erection.

Therefore, methods, systems, and devices that offer improved efficacy in regard to both short-term and long-term outcomes for erectile dysfunction would be advantageous, desirable and useful.

### SUMMARY OF THE DISCLOSURE

Embodiments of the disclosures made herein are directed to methods, systems, and devices that leverage the underlying treatment concept of propagating wave energy therapy to offer improved efficacy in regard to both short-term and long-term outcomes for erectile dysfunction. More specifically, embodiments of the disclosures made herein advantageously provide an approach to more effectively treat erectile dysfunction with faster, longer-lasting, and more reliable results. To this end, embodiments of the disclosures made herein are advantageously configured to apply propagating energy waves directly to the penis while the penis is maintained within a penis treatment device filled with a fluid that supports efficient travel of propagating waves through such fluid. The penis treatment device may be configured as a negative-pressure penis erection device (i.e., a penis erector). The penis treatment device also may be configured to provide a containment space for a penis and a fluid that supports efficient travel of propagating waves through such fluid, but without being configured for promoting an erection via negative pressure.

One objective of methods, systems, and devices in accordance with the disclosures made herein is to assist in regeneration of the male reproductive tissues and organs to correct at least partially degenerative conditions resulting from aging or disease. Another objective of methods, systems, and devices in accordance with the disclosures made herein is to stimulate the healing process of the male reproductive system after corrective surgery in cases where surgery is required to repair a defect in reproductive tissue or organs. Still another objective methods, systems, and devices in accordance with the disclosures made herein is to stimulate tissue revascularization and blood flow effectively to improve either performance or sensitivity to sexual contact thereby enhancing sexual experience for sexual partners.

In one or more embodiments of the disclosures made herein, a device for treatment of a penis of a patient comprises a main body having a central passage extending between a first opening at a first end portion of the main body and a second opening at a second end portion of the main body, a first sealing body attached to the first end portion of the main body, and a second sealing body attached to the second end portion of the main body. Material characteristics of at least a portion of the main body cause reflection of propagating energy waves that impinge upon the at least a portion of the main body. The first sealing body has a central passage for enabling at least a distal end portion of the penis to be positioned within the central passage of the main body. The first sealing body has a fluid-tight seal with the main body and is adapted to form a fluid-tight seal with a body portion of the patient adjacent to the penis. The second sealing body inhibits leakage of fluid through the second opening of the main body from within the central passage of the main body. Material characteristics of the second sealing body enable propagating energy waves to propagate therethrough.

In one or more embodiments of the disclosures made herein, a device for treatment of a penis of a patient comprises a penis erector and a containment body. The penis erector includes a penis-receiving body adapted for enabling the penis to be disposed within an interior space thereof. The penis erector is adapted for enabling a negative pressure environment to be generated within the interior space of the penis-receiving body to cause the penis disposed within the penis-receiving body and exposed to the negative pressure environment to become erect. The containment body has a central passage extending between a first opening at a first end portion of the containment body and a second opening at a second end portion of the containment body. A distal end portion of the penis erector is disposed within the central passage through the first opening. The first end portion of the containment body is engaged with a mating portion of the penis erector to create a reflection chamber between the penis erector and the containment body. The second end portion of the containment body is adapted for engaging a mating portion of an emitter body of a propagating energy wave source for enabling waves to be emitted into the reflection chamber through the second opening from the propagating energy wave source. Material characteristics of the containment body cause the propagating energy waves that impinge upon a surface of the containment body defining the central passage of the containment body to be reflected therefrom.

In one or more embodiments of the disclosures made herein, a system for treatment of a penis of a patient comprises a penis treatment device and a propagating energy wave source. The penis treatment device includes a main body having a central passage extending between a first opening at a first end portion of the main body and a second opening at a second end portion of the main body, a first sealing body attached to the first end portion of the main body, a second sealing body attached to the second end portion of the main body, and a propagating energy wave module attached to the second end portion of the main body. Material characteristics of at least a portion of the main body cause reflection of waves that impinge upon the at least a portion of the main body. The first sealing body has a central passage for enabling at least a distal end portion of the penis to be positioned within the central passage of the main body. The first sealing body has a fluid-tight seal with the main body and is adapted to form a fluid-tight seal with a body portion of the patient adjacent to the penis. The second sealing body inhibits leakage of fluid through the second opening of the main body from within the central passage of the main body. Material characteristics of the second sealing body enable propagating energy waves to propagate therethrough. The propagating energy wave source is located in front of the second opening of the main body.

In one or more embodiments of the disclosures made herein, a method for treating a penis of a patient comprises the steps of: positioning the penis within a penis-receiving space of a penis treatment system, causing the penis-receiving space to become at least partially filled with an energy wave propagating media, and emitting propagating energy waves into the penis-receiving space after causing the penis-receiving space to become at least partially filled with the energy wave propagating media whereby the penis within the penis-receiving body is subjected to the propagating energy waves. The penis-receiving space is at least partially encompassed by a layer of material having material characteristics causing the propagating energy waves that impinge upon the layer of material to be reflected therefrom.

In one or more embodiments, the main body includes a first fluid flow body adjacent the first end portion and a second fluid flow body adjacent the second end portion.

In one or more embodiments, the second end portion of the main body has a larger cross-sectional area than a cross-sectional area of a central portion of the main body and a cross-sectional area of the first end portion of the main body.

In one or more embodiments, the cross-sectional area of the central portion of the main body is approximately the same as the cross-sectional area of the first end portion of the main body.

In one or more embodiments, the first sealing body is at least one of flexible and resilient.

In one or more embodiments, the first sealing body is made from one of an elastomeric material, a synthetic rubber material, and a natural rubber material.

In one or more embodiments, the device further comprised a propagating energy wave source positioned one of within the central passage of the main body and in front of the second opening of the main body.

In one or more embodiments, a centerline longitudinal axis of the main body extends colinearly with a shock wave emittance axis of the propagating energy wave source.

In one or more embodiments, the device further comprises a propagating energy wave module attached to the second end portion of the main body, where the propagating energy wave module includes a propagating energy wave source located in front of the second opening of the main body.

In one or more embodiments, the first sealing body is located between the propagating energy wave source and the main body.

In one or more embodiments, the first end portion has a smaller cross-sectional area than a cross-sectional area of a central portion of the containment body.

In one or more embodiments, the first end portion of the containment body being engaged with the mating portion of the penis erector includes the first end portion of the containment body being in a sealed engagement with the mating portion of the penis erector.

In one or more embodiments, the penis erector includes a sealing body for providing a fluid-tight seal between the penis-receiving body and a body of the patient.

In one or more embodiments, the first end portion of the containment body being engaged with the mating portion of the penis erector includes the first end portion of the containment body being engaged with the sealing body.

In one or more embodiments, the penis erector includes a sealing body for providing a fluid-tight seal between the penis-receiving body and a body of the patient.

In one or more embodiments, the first end portion of the containment body being engaged with the mating portion of the penis erector includes the first end portion of the containment body being engaged with the penis erector at a location adjacent to an interface between the penis-receiving body and the sealing body.

In one or more embodiments, the fluid surrounding the penis in the penis-receiving space is liquid.

In one or more embodiments, emitting propagating energy waves into the penis-receiving space includes emitting the propagating energy waves whereby a shock wave emittance axis of the propagating energy waves extends colinearly with a centerline longitudinal axis of the penis-receiving space.

In one or more embodiments, the penis-receiving space is defined by a penis-receiving body of a penis erector, at least a portion of the penis-receiving body is within a central passage of a containment body, and emitting propagating energy waves into the penis-receiving space includes emitting the propagating energy waves into a reflection chamber jointly defined by the penis-receiving body and the containment body.

In one or more embodiments, causing the penis-receiving space to become at least partially filled with the energy wave propagating media includes causing the penis-receiving space to become at least partially filled with the energy wave propagating media in a manner whereby a negative pressure environment is generated within the penis-receiving space.

The method of claim 23, wherein said causing the penis-receiving space to become at least partially filled with the energy wave propagating media includes causing a volume of air within the penis-receiving space to be reduced.

In one or more embodiments, wherein the energy wave propagating media is liquid.

These and other objects, embodiments, advantages and/or distinctions of the present invention will become readily apparent upon further review of the following specification, associated drawings and appended claims.

### DEFINITIONS AND ASSOCIATED DISCLOSED FUNCTIONALITIES

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to one or more embodiments of the disclosures made herein is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nanoseconds (ns) up to some milliseconds (ms). Very fast pressure pulses are called shock waves. Shock waves used in medical applications do have amplitudes above 0.1 MPa and rise times of the amplitude can be below 1000 ns, preferably at or below 100 ns.

“Senescence” refers to loss of a cell's power of division and growth, also the condition or process of deterioration with age. It can refer to either cellular senescence or to senescence of the whole organism. Senescence is a process in which cells reach permanent growth arrest without the death of cells as the whole cell division process stops. A senescent cell is alive but cannot divide, but also has an active metabolism and secretes signaling molecules to communicate with other cells which can be beneficial, such as during wound healing, or detrimental, in the case of chronic inflammation. Senescence is an example of a cellular response that may be mitigated (e.g., via promoting generation of new cells) in response to stimulation of cells in accordance with embodiments of the disclosures made herein.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Washington 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

“Telomeres” play a central role in cell fate and aging by adjusting the cellular response to stress and growth stimulation on the basis of previous cell divisions and DNA damage. At least a few hundred nucleotides of telomere repeats must “cap” each chromosome end to avoid activation of DNA repair pathways. Repair of critically short or “uncapped” telomeres by telomerase or recombination is limited in most somatic cells and apoptosis or cellular senescence is triggered when too many “uncapped” telomeres accumulate. Telomerase and recombinations thereof are examples of proteins that may be released in response to stimulation of cells in accordance with embodiments of the disclosures made herein that promote regulation and lengthening of telomeres such as, for example, via activation of DNA repair pathways.

Waves/wave fronts described as being “focused” or “having focusing characteristics” means in the context of the present invention that the respective waves or wave fronts are traveling and increase their amplitude in direction of the focal point. Per definition, the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow diagram view of a method for treatment of a penis in accordance with one or more embodiments of the disclosures made herein.

FIG. 2A is a side view showing a penis treatment system in accordance with a first embodiment of the disclosures made herein.

FIG. 2B is a fragmentary, cross-sectional view of the penis treatment system of FIG. 2A taken along an in-page plane.

FIG. 2C is a color drawing with a diagrammatic cross-sectional view depicting relative propagating wave energy intensity for a penis treatment device configured similarly to the penis treatment device of FIG. 2A.

FIG. 2D is a color drawing with a diagrammatic cross-sectional view depicting relative propagating wave energy intensity delivered into a penis in fluid within a central passage of the penis treatment device of FIG. 2C.

FIG. 3A is a side view showing a penis treatment device of the penis treatment system shown in FIG. 2A as implemented in a penis treatment system in accordance with one or more embodiments of the disclosures made herein.

FIG. 3B is a fragmentary, cross-sectional view of the penis treatment device of FIG. 3A taken along an in-page plane.

FIG. 4 is a side view showing a penis treatment system in accordance with a second embodiment of the disclosures made herein.

FIG. 5 is a fragmentary, cross-sectional view of the penis treatment system of FIG. 4 taken along an in-page plane.

FIG. 6 is a side view showing a penis treatment system in accordance with a third embodiment of the disclosures made herein.

FIG. 7 is a fragmentary, cross-sectional view of the penis treatment system of FIG. 6 taken along an in-page plane.

FIG. 8 is a partial, cross-sectional view showing a penis treatment system in accordance with one or more embodiments of the disclosures made herein, where the partial, cross-sectional view is taken along an in-page plane.

FIG. 9 is a diagrammatic view of a pressure pulse/shock wave (PP/SW) generator showing focusing characteristics of transmitted acoustic pressure pulses.

FIG. 10 is a diagrammatic view of a pressure pulse/shock wave generator with plane wave characteristics.

FIG. 11 is a diagrammatic view of a pressure pulse shock wave generator (i.e., a shock wave head of a propagating energy wave source) with divergent wave characteristics.

FIG. 12 is a diagrammatic view of an exemplary acoustic shock wave apparatus in accordance with one or more embodiments of the disclosures made herein.

### DETAILED DESCRIPTION

Embodiments of the disclosures made herein are directed to methods, systems, and devices that leverage the underlying treatment concept of propagating wave energy therapy (e.g., acoustic shockwave therapy) to offer improved efficacy in regard to both short and long term outcomes for erectile dysfunction. Embodiments of the disclosures made herein are advantageously configured to apply propagating energy waves (e.g., acoustic shock waves) directly to the penis while the penis is surrounded by a fluid that supports efficient travel of propagating waves through such fluid. In some embodiments, pressure pulses in the form of acoustic shock waves or other types of propagating energy wave may be used in combination with negative pressure based blood flow enhancement of the penis to provide treatment of erectile dysfunction and other related/associated conditions. For example, during treatment with such propagating wave energy (i.e., energy provided by exposure to propagating energy waves), the penis may be maintained in an erect state through use of negative-pressure such as via a penis treatment device configured to enable generation of negative pressure within a space in which a penis is located during treatment (i.e., a penis erector). As is well known in the art, placement of the penis in the negative pressure environment generated using a fluid (e.g., water) that surrounds the penis produces an erection of the penis for enabling transmission of propagating wave energy with limited attenuation and optimal penetration of such propagating wave energy into the penis tissue.

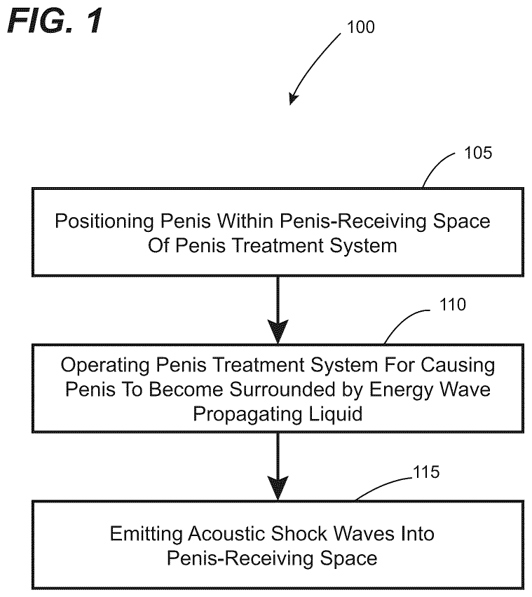

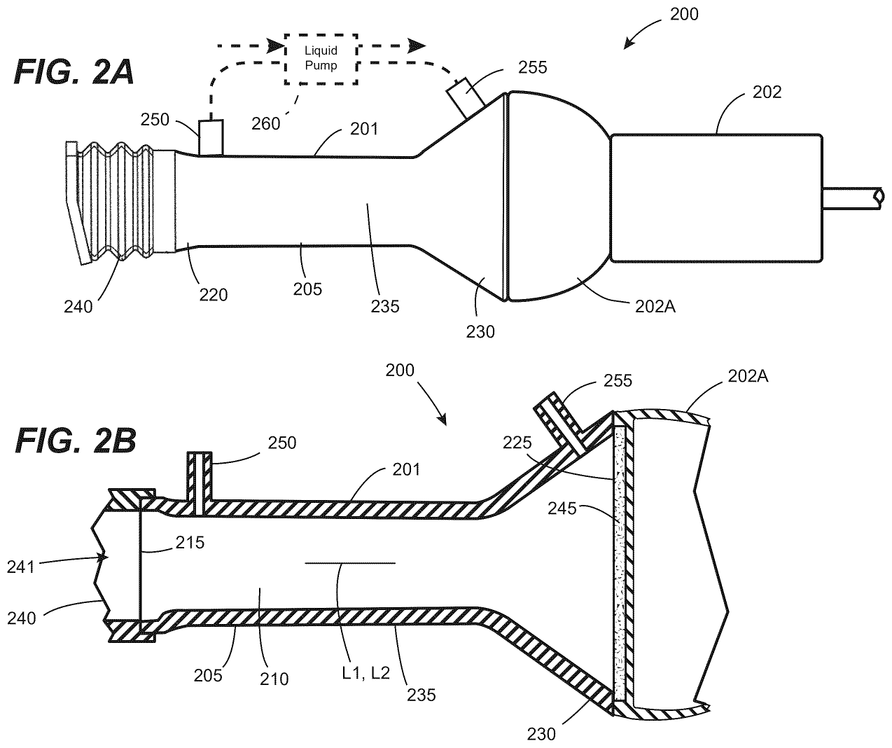

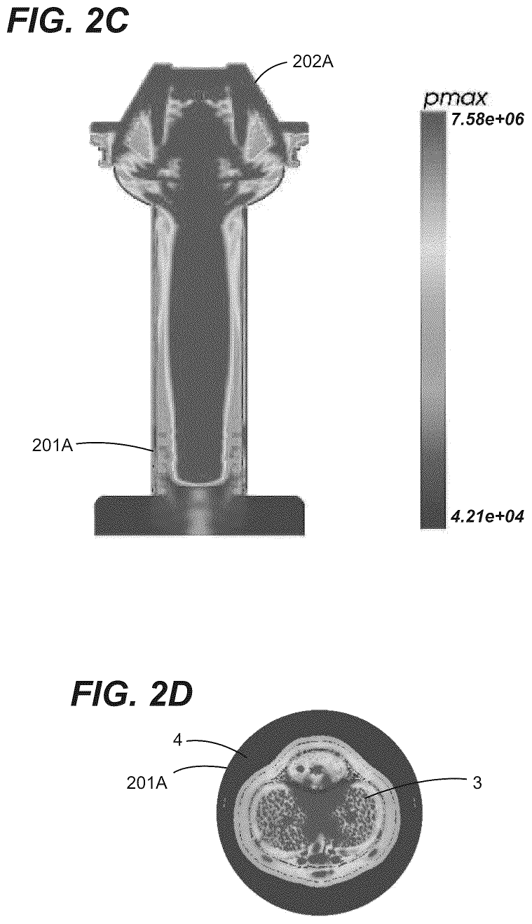

Referring to FIG. 1, a method for treatment of a penis, such as related to treatment of erectile dysfunction, is shown—i.e., method 100. The method 100 may be implemented utilizing devices and systems in accordance with embodiments of the disclosures made herein—e.g., as disclosed in reference to FIGS. 2-8. However, the method 100 is not necessarily limited to any particular device, apparatus, or system for its implementation.

The method 100 includes a step 105 for positioning a penis within a penis-receiving space of a penis treatment system and a step 110 for operating the penis treatment system for causing the penis to become surrounded by a fluid that supports efficient travel of propagating waves through such fluid. As discussed below in greater detail, causing the penis to become erect may include causing a negative pressure environment to be generated within the penis-receiving space such as by causing a volume of fluid within the penis-receiving space surrounding the penis within an enclosed space to be reduced without a corresponding reduction of volume of the interior space. It is disclosed herein that the fluid may be liquid or air or a combination thereof. In preferred embodiments, the penis is entirely surrounded or effectively surrounded by an energy wave propagating media that is liquid (e.g., water, saline solution, or other aqueous material composition exhibiting energy wave propagating with negligible attenuation). As is known in the art, the negative pressure (i.e., a vacuum condition) causes a pressure differential between the inside and outside of the penis-receiving space via extraction of fluid surrounding the penis (an interior space-filling fluid) thereby inducing the penis to fill with blood and become erect (in the case of the penis being flaccid) or more erect (in the case of a penis that is partially erect).

The method 100 includes a step 115 for emitting acoustic shock waves (or other type of propagating energy waves) into the penis-receiving space whereby the penis within the penis-receiving body, which may or may not be erect, is exposed to the acoustic shock waves. The acoustic shock waves (or other type of propagating energy waves) provides for stimulation of cells of penile tissue and resulting cellular regeneration of such tissue such as via triggering of a cellular response that causes release of growth factors/hormones, triggering of a cellular response that causes release of telomeres, triggering of a cellular response that mitigates senescence, and triggering of one or more cellular responses that causes other cellular regenerative mechanisms beneficial to reducing adverse effects of erectile dysfunction.

Such emitting of the acoustic shock waves and result exposure of the penis thereto may be performed in a variety of manners with respect to primary direction of emission from an acoustic shock wave source. In some embodiments, a shock wave emittance axis of the acoustic shock waves from an acoustic shock wave source extends colinearly with a centerline longitudinal axis of the penis-receiving space (e.g., along a common reference axis). In some embodiments, a shock wave emittance axis of the acoustic shock waves from an acoustic shock wave source extends perpendicular to a centerline longitudinal axis of the penis-receiving space. In some embodiments, the acoustic shock waves are emitted in a focused manner and may be divergent. In some embodiments, the acoustic shock waves are emitted in an unfocused manner and may be divergent.

In some embodiments, the penis-receiving space may be partially or fully encompassed by a layer of material (e.g., a structural wall or surface layer of a wall) having material characteristics causing the acoustic shock waves that impinge upon the layer of material to be reflected therefrom with a high level of retained energy (i.e., negligible attenuation). Such reflection of the acoustic shock waves is beneficial to maintaining energy associated with the acoustic shock waves available for acting on the penis as opposed to being released into an ambient environment surrounding the penis treatment system or otherwise diminishing such energy and the beneficial treatment aspects thereof. Accordingly, the propagating energy waves are introduced into the penis-receiving space from the propagating energy wave source and are reflected around and into the penis to thereby result in the penis being exposed to the propagating energy waves in a more homogeneous (e.g., omnidirectional) manner than without such intended reflection.

Without wishing to be limited to such disclosure, a skilled person will understand that examples of material providing reflection of acoustic shock waves in a manner that maintains a high level of energy of acoustic shock waves and other types of propagating energy waves (i.e., negligible attenuation) include, but are not limited to, metallic material, material with a metallic layer, certain crystalline material (e.g., glass), polymeric material having high crystallinity and/or density, and the like. More generally, material providing reflection of acoustic shock waves offer high impedance and, thus, reflect sound waves. A skilled person will also understand that thickness is another material characteristic that impacts acoustic shock wave reflectivity and attenuation.

Referring now to FIGS. 2-8, various aspects of implements for carrying out treatment of a penis in accordance with one or more embodiments of the disclosures made herein are discussed. In some instances, such an implement is a system suitable for carrying out the disclosed treatment of a penis such as via the method 100 for treatment of a penis discussed above in reference to FIG. 1. In other instances, such an implement is a device that is used in combination with other devices, apparatuses, and/or systems for carrying out the treatment of a penis as disclosed herein. Regardless of the specific implement, the underlying objective is to apply propagating energy waves (e.g., acoustic shock waves) directly to a penis while the penis is entirely surrounded or effectively surrounded by a fluid that supports efficient travel of propagating waves through such fluid to offer improved erectile dysfunction treatment efficacy in regard to both short and long term outcomes for erectile dysfunction. Effectively surrounded is defined herein to include the condition where the penis is surrounded by a liquid (e.g., water) containing negligible/acceptable amount of gaseous air bubbles and/or adjacent pockets of air. Optionally, a negative pressure may be applied to the penis via the fluid through application of negative pressure for causing the penis to be maintained in an erect state during treatment with the propagating wave energy.

FIGS. 2A and 2B show a penis treatment system in accordance with a first embodiment of the disclosures made herein—i.e., system 200. The system 200 includes a penis treatment device 201 (i.e., a negative-pressure penis erecting device) and a propagating energy wave source 202. Although acoustic shock waves are a preferred type of propagating energy wave (i.e., pressure pulses) useful for the treatment of a penis in accordance with embodiments of the disclosures made herein, a skilled person will understand that other types of propagating energy waves (e.g., radial pressure waves, acoustic pneumatic waves, ultrasonic waves, and the like) may be useful for treatment of a penis in accordance with embodiments of the disclosures made herein. It is well known in the art that propagating energy waves may be provided via electromagnetic or piezo electric, ballistic or electro-hydraulic sources or generators, and the like.

The penis treatment device 201 includes a main body 205 having a central passage 210 extending between a first opening 215 at a first end portion 220 of the main body 205 and a second opening 225 at a second end portion 230 of the main body 205. The central passage 210 is a penis-receiving space of the penis treatment device 201. Material characteristics of at least a portion of the main body 205 cause reflection of propagating energy waves (e.g., acoustic shock waves) that impinge upon the at least a portion of the main body, thereby limiting energy attenuation of the propagating energy waves.

The second end portion 230 of the main body 205 may have a larger cross-sectional area than a cross-sectional area of a central portion 235 of the main body 205 and a cross-sectional area of the first end portion 220 of the main body 205. The cross-sectional area of the central portion 235 of the main body 205 may be approximately the same as the cross-sectional area of the first end portion 220 of the main body 205 (e.g., a uniform cross-sectional diameter). The central portion 235 of the main body 205 may have a tapered arrangement where the cross-sectional area of the main body 205 continually increases in cross-sectional area from the first end portion 220 to the second end portion 230. The central passage 210 of the main body 205 may have a round cross-sectional shape over all or a portion of its length. The respective cross-sectional areas of the main body 205 may be at least partially dependent upon overall size of the main body 205 for accommodating a required range of penis sizes and a size (e.g., diameter) of an emitter portion 202A of the propagating energy wave source 202.

A first sealing body 240 is attached to the first end portion 220 of the main body 205. The first sealing body 240 has a central passage 241 that extends contiguously with the central passage 210 of the main body 205 for enabling at least a distal end portion of a penis of a patient to be positioned within the central passage 210 of the main body 205. The first sealing body 240 has a fluid-tight seal with the main body 205 and is adapted to form a fluid-tight seal with a body portion of the patient adjacent to the penis. In the context of the disclosures made herein, a “patient” is an example of a type of user and not all users are a patient.

In some embodiments, the first sealing body 240 may be in the form of a gaiter-type seal. Gaiter-type seals are well known in the context of penis erectors as having a bellow-like configuration that allows for displacement of the main body of a penis erector without breaking a fluid-tight seal between the penis erector and a body of the patient of the penis erector. Accordingly, the first sealing body 240 is preferably made from a material that is flexible and/or resilient such as, for example, an elastomeric material, a synthetic rubber material, and a natural rubber material. The functionality of a fluid tight seal in accordance with the disclosures herein refers to a seal that inhibits or limits the flow of a fluid (e.g., gaseous and/or liquid) within the central passage of the main body 205 sufficiently to enable operation of the penis treatment device 201 for a required/desired duration of time (e.g., the length of a penis treatment session using the penis treatment device 201).

A second sealing body 245 is attached to the second end portion 230 of the main body 205. The second sealing body 245 inhibits leakage of fluid through the second opening 230 of the main body 205 from within the central passage 210 of the main body 205. The second sealing body 245 may be made from a material that is flexible and/or resilient such as, for example, an elastomeric material, a synthetic rubber material, and a natural rubber material. Material characteristics of the second sealing body 245 enable propagating energy waves to pass through the second sealing body 245 (e.g., a sound-permeable membrane). Accordingly, such material of the second sealing body 245 will offer water impermeability in combination with minimal or negligible attenuation of propagating energy waves as useful in embodiments of the disclosures made herein.

The propagating energy wave source 202 is engaged with the main body 205 at its second end portion 230 in front of the second opening 225. The emitter portion 202A of the propagating energy wave source 202 (e.g., an electrohydraulic reflector) is engaged with (e.g., attached to) the main body 205 at its second end portion 230 in front of the second opening 225. The second sealing body 245 is located between the propagating energy wave source 202 and the second opening 225 to inhibit fluid transfer between the central passage 210 of the main body 205 and the propagating energy wave source 202. In some embodiments, the second sealing body 245 may be a coupling membrane of the propagating energy wave source 202. In some embodiments, the emitter portion 202A of the propagating energy wave source 202 may be removably (e.g., threadedly or otherwise interlockedly) engaged with the main body 205 to permit selective attachment to and detachment from disposable and/or re-usable instances of the penis treatment device 201.

In some embodiments, treatment of a penis is performed while the penis is erect. However, in view of treatment of erectile dysfunction being a preferred application for devices, systems and methods in accordance with the disclosures made herein, it may be impossible or impractical for such erect state of the penis to be naturally achieved during a treatment session. To facilitate an erect state of the penis during a treatment session and to facilitate evacuation of air bubbles/pockets from within the central passage 210, the main body 205 may include a first fluid flow body 250 adjacent its first end portion 220 and a second fluid flow body 255 adjacent its second end portion 230. To this end, the first and second fluid flow bodies 250, 255 may be attached to a liquid pump apparatus 260 for managing flow of a fluid (e.g., water) into and out of the central passage 210 of the main body 205 for causing a penis within the central passage 210 (i.e., a penis-receiving space) to become erect. For example, the liquid pump apparatus 260 may be used for managing flow of a fluid into and out of the central passage 210 of the main body 205 for causing pressure within central passage 210 to become less than an ambient pressure surrounding the main body 205. Accordingly, in accordance with the well-known technique of using negative pressure to facilitate an erection, the pressure within central passage 210 surrounding the penis becoming less than the ambient pressure surrounding the main body 205 will cause increased blood flow into the penis and, thus, erection of the penis. Beneficially, as discussed above, the first sealing body 240 being in the form of a gaiter-type seal or the like will allow for displacement of the main body 205 relative to a body of a person being treated during generation of such negative pressure without breaking a fluid-tight seal between the first sealing body 240 and the person's body.

As a skilled person will understand, when the first sealing body 240 is a gaiter-type seal or the like that allows for displacement of the main body 205 relative to the body of the person being treated, the fluid flow body and fluid pump arrangement may be replaced with a one-way fluid evacuation valve. The combination of the gaiter-type seal and the one-way fluid evacuation valve permits the negative pressure to be generated through manual reciprocating displacement of the main body 205 relative to the body of the person being treated. For example, such an arrangement and functionality of a gaiter-type seal and one-way fluid evacuation valve is disclosed by the penile rigidity device (penis erector) in U.S. Pat. No. 9,039,598, which is incorporated herein in its entirety by reference.

Referring to FIGS. 2C and 2D, relative propagating wave energy intensity for a penis treatment device 201A configured in accordance with the penis treatment device 201 of FIG. 2A is shown. Propagating wave energy is emitted into the central passage 210 from an emitter portion 202A of a propagating energy wave source. As shown in FIG. 2D, a penis 3 may be disposed in a liquid 4 within a central passage 210 of the penis treatment device 201A for enabling treatment with propagating wave energy.

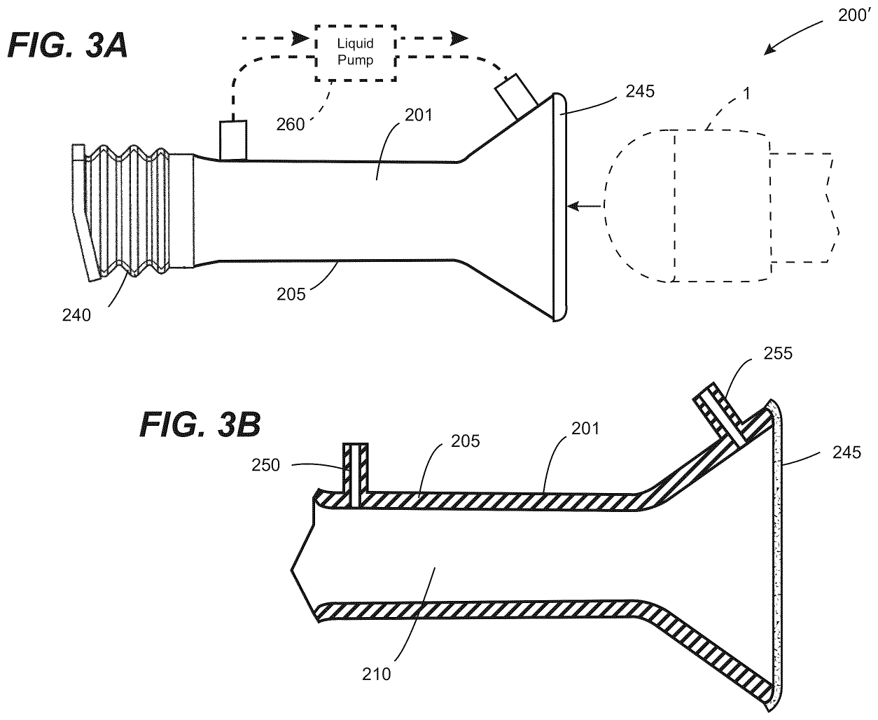

Referring to FIGS. 3A and 3B, the penis treatment device 201 of the penis treatment system 200 shown in FIGS. 2A and 2B may be separately implemented as a penis treatment device in accordance with embodiments of the disclosures made herein. To this end, in FIGS. 3A and 3B, the propagating energy wave source 202 of the of the penis treatment system 200 is omitted. A propagating energy wave emitter 1 (e.g., an acoustic shock electrohydraulic reflector) may be engaged directly with the second sealing body 245 to enable propagating energy waves to be emitted into the central passage 210 for treating a penis within the central passage 210. Thus, as shown in FIG. 3A, the penis treatment device 201 is implemented in a penis treatment system 200′ which is characterized as comprising a propagating energy wave source that is not integral with the penis treatment device 201.

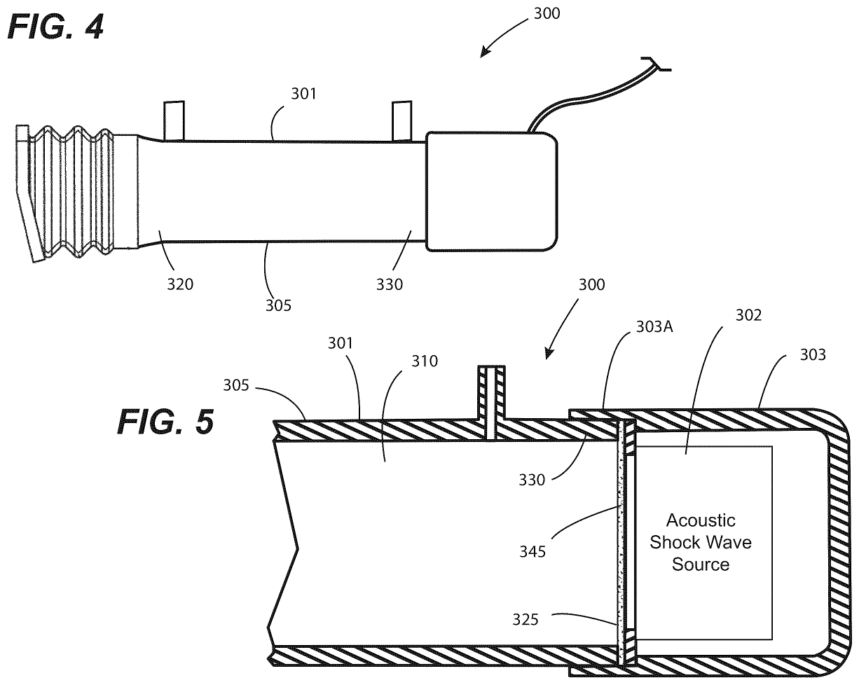

Turning now to FIGS. 4 and 5, a penis treatment system in accordance with a second embodiment of the disclosures made herein is shown—i.e., system 300. The system 300 has the same underlying applicability and operability as the penis treatment (i.e., the system 200) discussed above in reference to FIGS. 2A and 2B. Specifically, the penis treatment system 300 similarly offers treatment of penile ailments such as erectile dysfunction through application of propagating energy waves (e.g., acoustic shock waves) directly to a penis while the penis is entirely surrounded or effectively surrounded by a fluid that supports efficient travel of propagating waves through such fluid to offer improved erectile dysfunction treatment efficacy in regard to both short and long term outcomes for erectile dysfunction. Optionally, the fluid may be used to exert a negative pressure on the penis such that the penis is maintained in an erect state during treatment with the propagating wave energy. The key difference between the system 200 and the system 300 is the implementation of the source of the propagating energy waves. For conciseness of disclosure, only the aspects of the system 300 that are structurally and/or operationally different than that of the system 200 will be described. Components of the system 300 that are similar to components of the system 200 will be designated by similar reference numerals—e.g., main body 305 of the system 300 correspond to main body 205 of the system 200.

The system 300 includes a penis treatment device 301 and a propagating energy wave source 302. The penis treatment device 301 includes a main body 305 having a central passage 310 extending between a first end portion 320 of the main body 305 and a second end portion 330 of the main body 305. The central passage 310 is a penis-receiving space of the penis treatment device 301. The shock wave source 302 resides within an acoustic shock wave source housing 303. An engagement portion 303A of the propagating energy wave source housing 303 is attached to second end portion 330 of the main body 305 of the penis treatment device 301 in front of a second opening 325 of the main body 305. A second sealing body 345 is located between the propagating energy wave source 302 and the second opening 325 for providing a fluid-tight seal therebetween to inhibit fluid transfer between the central passage 310 of the main body 305 and the propagating energy wave source 302. In some embodiments, the engagement portion 303A of the propagating energy wave source housing 303 may be removably (e.g., threadedly or otherwise interlockedly) engaged with the main body 305 to permit selective attachment and detachment of the propagating energy wave source 302 to disposable or re-usable instances of the penis treatment device 301.

The propagating energy wave source 302 may be any type of source of propagating energy waves that offer propagating energy waves in a form suitable for facilitating treatment of a penis in accordance with embodiments of the disclosures made herein. Although acoustic shock waves are a suitable type of propagating energy wave (i.e., pressure pulses) useful for the treatment of a penis in accordance with embodiments of the disclosures made herein, a skilled person will understand that other types of propagating energy waves (e.g., radial pressure waves, acoustic pneumatic waves, ultrasonic waves, and the like) may be useful for treatment of a penis in accordance with embodiments of the disclosures made herein. In preferred embodiments, the propagating energy wave source 302 may be a self-contained source (e.g., without an electrohydraulic reflector) of propagating energy waves that are emitted from the propagating energy wave source 302 through the second sealing body 345 and into fluid (e.g., water) within the central passage 310 of the main body 305. In some embodiments, the second sealing body 345 may be omitted such as when the propagating energy wave source includes an integral seal or sealing means for providing a water-tight to an interior space of the propagating energy wave source 302 and/or the interior space of the propagating energy wave source housing 303.

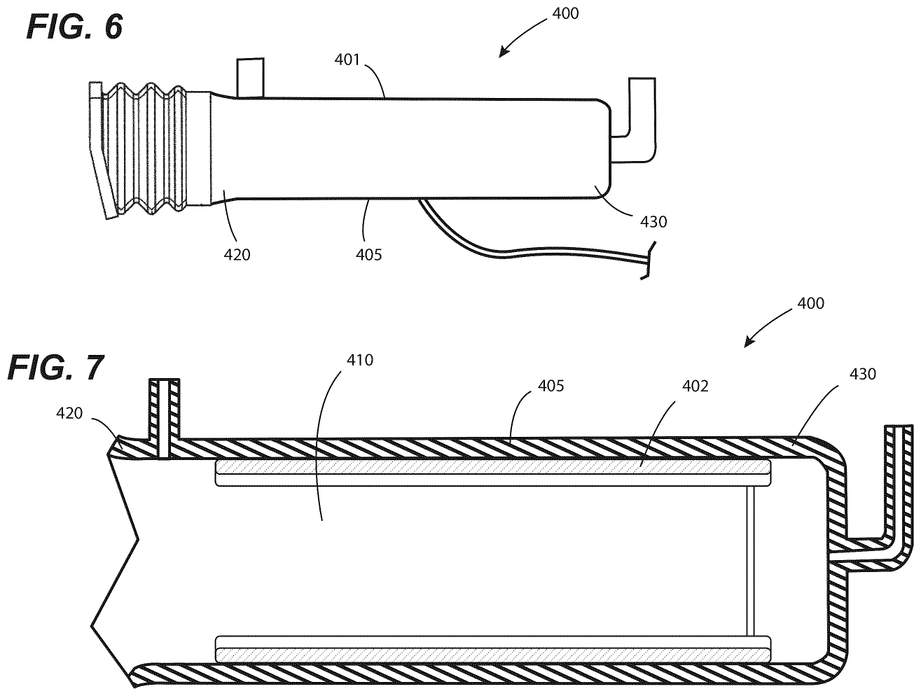

FIGS. 6 and 7 show a penis treatment system in accordance with a third embodiment of the disclosures made herein—i.e., system 400. The system 400 has the same underlying applicability and operability as the penis treatment system discussed above in reference to FIGS. 2A and 2B (i.e., the system 200). Specifically, the penis treatment system 400 similarly offers treatment of penile ailments such as erectile dysfunction through application of propagating energy waves (e.g., acoustic shock waves) directly to a penis while the penis is entirely surrounded or effectively surrounded by a fluid that supports efficient travel of propagating waves through such fluid to offer improved erectile dysfunction treatment efficacy in regard to both short and long term outcomes for erectile dysfunction. Optionally, the fluid may be used to exert a negative pressure on the penis such that the penis is maintained in an erect state during treatment with the propagating wave energy. The key difference between the system 200 and the system 400 is the implementation of the source of the propagating energy waves. For conciseness of disclosure, only the aspects of the system 400 that are structurally and/or operationally different than that of the system 200 will be described. Components of the system 400 that are similar to components of the system 200 will be designated by similar reference numerals—e.g., main body 405 of the system 400 correspond to main body 205 of the system 200.

The system 400 includes a penis treatment device 401 having a main body 405 with a central passage 410. The central passage 410 extends between a first end portion 420 of the main body 405 and a second end portion 430 of the main body 405. The central passage 410 is a penis-receiving space of the penis treatment device 401. A propagating energy wave source 402 is located within the central passage 410. The propagating energy wave source 402 may be attached to or otherwise engaged with one or more walls of the main body 405 that define the central passage 410. In some embodiments, the propagating energy wave source 402 extends along at least a portion of the length of the central passage 410. The propagating energy wave source 402 may offer generation of propagating energy waves from multiple source locations and offer more direct/abundant impingement of propagating energy wave source upon the penis during treatment. In one or more embodiments, the propagating energy wave source 402 may utilize be a solid-state type of propagating energy wave source. For example, piezoelectric propagating energy wave sources are disclosed in U.S. Pat. No. 5,119,801 and in United States Patent Application Publication 2004/0167445, which are incorporated herein in their entirety by reference.

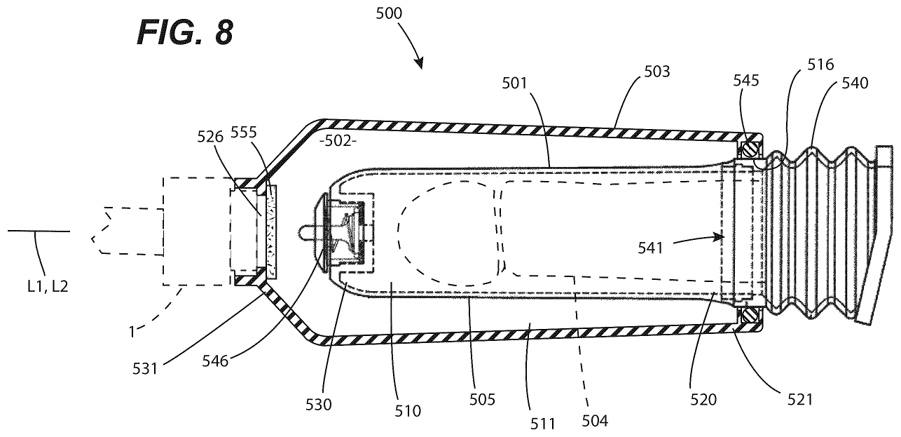

FIG. 8 shows a penis treatment device in accordance with one or more embodiments of the disclosures made herein—i.e., device 500. The device 500 includes a penis erector 501 (i.e., a negative-pressure penis erecting device) and containment body 503. The penis erector 501 includes a main body 505 having a central passage 510 extending between a first end portion 520 of the main body 505 and a second end portion 530 of the main body 505. The central passage 510 is a penis-receiving space of the penis erector 501. In this respect, the main body 505 is a penis-receiving body adapted for enabling a penis 504 to be disposed within the central passage 510.

A first sealing body 540 is attached to the first end portion 520 of the main body 505. The first sealing body 540 has a central passage 541 that extends contiguously with the central passage 510 of the main body 505 for enabling at least a distal end portion of a penis of a patient (e.g., a patient) to be positioned within the central passage 510 of the main body 505. The first sealing body 540 has a fluid-tight seal with the main body 505 and is adapted to form a fluid-tight seal with a body portion of the patient adjacent to the penis.

The penis erector 501 includes a one-way fluid evacuation valve 546 that is integral with the main body 505. The one-way fluid evacuation valve 546 allows fluid to pass from the central passage 510 to the ambient environment but not in the opposite direction. The first sealing body 540 may be in the form of a gaiter-type seal. Gaiter-type seals are well known in the context or penis erectors as having a bellow-like configuration that allows for displacement of the main body of a penis erector without breaking a fluid-tight seal between the penis erector and a body of the patient of the penis erector. Accordingly, the first sealing body 540 is preferably made from a material that is flexible and/or resilient such as, for example, an elastomeric material, a synthetic rubber material, and a natural rubber material. The functionality of a fluid tight seal in accordance with the disclosures herein refers to a seal that inhibits or limits the flow of a fluid (e.g., gaseous and/or liquid) sufficiently for enabling operation of the penis erector 501 for a required/desired duration of time (e.g., the length of a penis treatment session using the penis erector 501). As is well known in the art, the combination of the one-way fluid evacuation valve 546 and the first sealing body 540 being in the form of a gaiter-type seal permits negative pressure to be generated within the central passage 510 of the main body 505 in response to manual reciprocating displacement of the main body 505 relative to the body of the person being treated.

The penis erector 501 may be commercially-available or proprietary. For example, the penis erector 501 is a prior art device whose construction and operation is described in detail in U.S. Pat. No. 9,039,598, which is incorporated herein in its entirety by reference. In general, the penis erector 501 enables managing flow of a fluid out of the central passage 510 of the main body 505 via a one-way valve for causing pressure within central passage 510 to become less than an ambient pressure surrounding the main body 505. Accordingly, in accordance with the well-known technique of using negative pressure to facilitate an erection, the pressure within central passage 510 becoming less than the ambient pressure surrounding the main body 505 causes increased blood flow into the penis and, thus, erection of the penis. Beneficially, the first sealing body 540 being in the form of a gaiter-type seal or the like will allow for displacement of the main body 505 relative to a body of a person being treated during generation of such negative pressure without breaking a fluid-tight seal between the first sealing body 540 and the person's body.

The containment body 503 has a central passage 511 extending between a first opening 516 at a first end portion 521 of the containment body 503 and a second opening 526 at a second end portion 531 of the containment body 503. In preferred embodiments, the central passage 511 of the containment body 503 has a round cross-sectional shape over all or a portion of its length. A distal end portion 501A of the penis erector 501 is disposed within the central passage 541 of the containment body 503 through the first opening 516. The first end portion 521 of the containment body 503 is engaged with a mating portion of the penis erector 501 to create a reflection chamber 502 between the penis erector 501 and the containment body 503. The second end portion 531 of the containment body 503 is adapted for engaging a mating portion of a propagating energy wave source 1 for enabling propagating energy waves to be emitted into the reflection chamber 502 through the second opening 526. Material characteristics of the containment body 503 cause the propagating energy waves that impinge upon the containment body 503 defining the central passage 511 of the containment body 503 to be reflected therefrom.

During use, the reflection chamber 502 may be filled with a fluid such as a gaseous material (e.g., air) and/or a liquid material (e.g., water). Similarly, during use, the central passage 510 of the penis erector 501 may be filled with a fluid such as a gaseous material (e.g., air) and/or a liquid material (e.g., water). In the case of the reflection chamber 502 being filled with a liquid material, the containment body 503 may include structural implements for enabling inclusion of such liquid within the reflection chamber 502. One such structural implement may be a first sealing body 545 of the containment body 503 that provides a fluid-tight seal with the penis erector 501. For example, the first sealing body 545 (e.g., an O-ring seal) may be engaged with the penis erector 501 at a location adjacent to an interface between the main body 505 and the first sealing body 540. Another such structural implement may be one or more fluid flow bodies 250, 255 as discussed above in reference to FIGS. 2A and 2B for enabling air to be expelled from within the reflection chamber 502 and to let liquid fill and/or be evacuated from within the reflection chamber 502. Still another such structural implement may be a second sealing body 555 of the containment body 503 that provides a fluid-tight seal at the second opening 526 of the containment body 503. Material characteristics of the second sealing body 555 enable propagating energy waves from the propagating energy wave source 1 to pass through the second sealing body 555 (e.g., a sound-permeable membrane). In this regard, such materials will offer water impermeability in combination with minimal or negligible attenuation of propagating energy waves as useful in embodiments of the disclosures made herein.

In preferred embodiments, treatment of tissue with propagating wave energy in accordance with one or more embodiments of the disclosures made herein may activate and/or normalize cellular function. To this end, such treatment may include activating an acoustic shock wave source (e.g., generator) to emit acoustic shock waves and subjecting target tissue of a patient to one or more treatments of exposure to acoustic shock waves. The acoustic shock waves provide pressure pulses each comprising a plurality of cycles of a positive pressure part and a negative pressure part. Subjecting the target tissue of the patient to the plurality of treatments of exposure to the acoustic shock waves is performed for causing stimulation of cells of the target tissue to initiate genetic expression of cells of the target tissue. Such stimulation of cells of the target tissue to initiate genetic expression may cause one or more of release of exosomes from cells of targeted (i.e., treated) tissue, activation of at least one cellular receptor, shedding of micro-vesicles from the cells of targeted tissue, and release of at least one of a protein, cytokines, and mRNA from the cells of targeted tissue into the extracellular matrix of the targeted tissue.

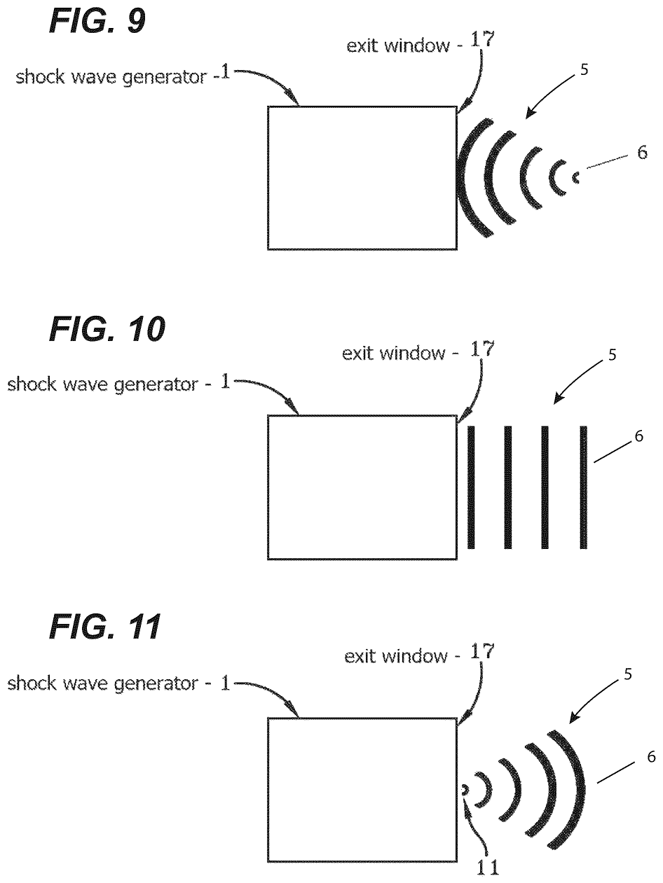

With reference to FIGS. 9-11, a variety of schematic views of acoustic shock waves or pressure pulses are described. The following description of the proper amplitude and pressure pulse intensities of the shock waves are provided along with a description of how the shock waves actually function. For the purpose of describing, the shock waves were used as exemplary and are intended to include all of the wave patterns discussed in the figures as possible treatment patterns. Specific details of acoustic shock waves and treatment therewith are disclosed in U.S. Pat. Nos. 7,470,240; 7,507,213; 7,601,127; 7,695,443; 8,257,282; 11,311,454; 11,389,371, which are incorporated herein in their entirety by reference.

FIG. 9 is a simplified depiction of a pressure pulse/shock wave (PP/SW) generator, such as a shock wave head, showing focusing characteristics of transmitted acoustic pressure pulses 5. Numeral 1 indicates the position of a generalized pressure pulse generator (i.e., propagating energy wave source 1), which generates pressure pulses 5 and, via a focusing element, focuses it outside the housing to treat diseases. The affected tissue or organ is generally located in or near the focal point which is located in or near position 6. At position 17 a water cushion or any other kind of exit window for the acoustical energy is located.

FIG. 10 is a simplified depiction of a pressure pulse/shock wave generator, such as a shock wave head, with plane wave characteristics. Numeral 1 indicates the position of a pressure pulse generator according to the present invention (i.e., propagating energy wave source 1), which generates pressure pulses 5 which are leaving the housing at the position 17, which may be a water cushion or any other kind of exit window. Somewhat even, also referred to herein as “disturbed,” wave characteristics can be generated, in case a paraboloid is used as a reflecting element, with a point source (e.g. electrode) that is located in the focal point of the paraboloid. The pressure pulses 5 will be transmitted into the patient's body via a coupling media such as, e.g., ultrasound gel or oil and their amplitudes will be attenuated with increasing distance from the exit window 17.

FIG. 11 is a simplified depiction of a pressure pulse shock wave generator (i.e., a shock wave head of a propagating energy wave source) with divergent wave characteristics. The divergent wave fronts may be leaving the exit window 17 at point 11 where the amplitude of the wave front is high. This point 11 could be regarded as the source point for pressure pulses 5. The pressure pulse source may be a point source, that is, the pressure pulse 5 may be generated by an electrical discharge of an electrode under water between electrode tips. However, the pressure pulses 5 may also be generated, for example, by an explosion, referred to as a ballistic pressure pulse. The divergent characteristics of the wave front may be a consequence of the mechanical setup.

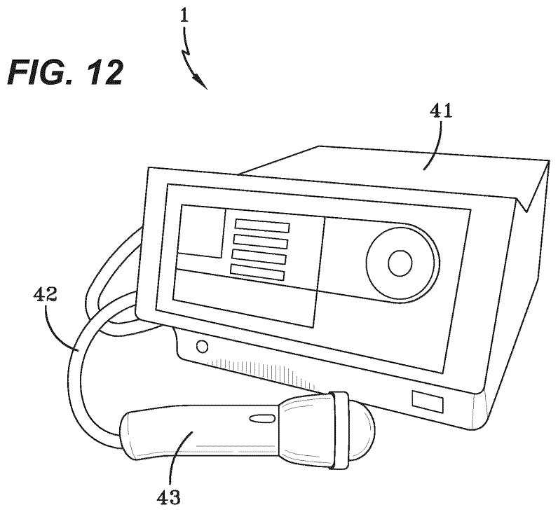

With reference to FIG. 12, an exemplary an acoustic shock wave apparatus 1 (i.e., a propagating energy wave source) is illustrated. The shock wave apparatus has a generator 41 connected by a flexible hose with fluid conduits extending from the shock wave generator 41 to an applicator 43 which transmits the acoustic waves when coupled to the skin by using a fluid or acoustic gel. The applicator 43 as illustrated has a body that enables a technician to hold the applicator 43 and as illustrated this applicator is an electrohydraulic device that is filled with fluid to facilitate the transmission of the shock waves. The fluid expands a flexible membrane in such a fashion that the membrane extends outwardly in a balloon shape fashion. As shown, this type of applicator 43 has a hydraulic spark generator using either focused or unfocused shock waves, preferably in a low energy level, less than the range of 0.01 milli joules (mJ)/mm² to 0.5 mJ/mm². In some embodiments, focused or unfocused shock waves may have an energy level up to at least 100.0 mJ/mm². The flexible hose 42 is connected to a fluid supply that fills the applicator 43 and expands the flexible membrane when filled. Alternatively, a ballistic, piezoelectric, or spherical acoustic shock wave device can be used to generate the desired waves.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in all its aspects. Although the invention has been described with reference to particular means, materials and embodiments, the invention is not intended to be limited to the particulars disclosed; rather, the invention extends to all functionally equivalent technologies, structures, methods and uses such as are within the scope of the appended claims.

What is claimed is:

1. A device for treatment of a penis of a patient, comprising:
a main body having a central passage extending between a first opening at a first end portion of the main body and a second opening at a second end portion of the main body, wherein material characteristics of at least a portion of the main body cause reflection of acoustic shock waves that impinge upon the at least a portion of the main body;
a first sealing body attached to the first end portion of the main body, wherein the first sealing body has a central passage for enabling at least a distal end portion of the penis to be positioned within the central passage of the main body, and wherein the first sealing body has a fluid-tight seal with the main body and is adapted to form a fluid-tight seal with a body portion of the patient adjacent to the penis; and
a second sealing body attached to the second end portion of the main body, wherein the second sealing body inhibits leakage of fluid through the second opening of the main body from within the central passage of the main body and wherein material characteristics of the second sealing body enable acoustic shock waves to propagate therethrough.

2. The device of claim 1, wherein the main body includes a first fluid flow body adjacent the first end portion and a second fluid flow body adjacent the second end portion.

3. The device of claim 1, wherein the second end portion of the main body has a larger cross-sectional area than a cross-sectional area of a central portion of the main body and a cross-sectional area of the first end portion of the main body.

4. The device of claim 3, wherein the cross-sectional area of the central portion of the main body is approximately the same as the cross-sectional area of the first end portion of the main body.

5. The device of claim 1, wherein the first sealing body is at least one of flexible and resilient.

6. The device of claim 1, wherein the first sealing body is made from one of an elastomeric material, a synthetic rubber material, and a natural rubber material.

7. The device of claim 1, further comprising:
an acoustic shock wave source positioned one of within the central passage of the main body and in front of the second opening of the main body.

8. The device of claim 7, wherein a centerline longitudinal axis of the main body extends colinearly with a shock wave emittance axis of the acoustic shock wave source.

9. The device of claim 7, wherein the second end portion of the main body has a larger cross-sectional area than a cross-sectional area of a central portion of the main body and a cross-sectional area of the first end portion of the main body.

10. The device of claim 9, wherein the cross-sectional area of the central portion of the main body is approximately the same as the cross-sectional area of the first end portion of the main body.

11. A device for treatment of a penis of a patient, comprising:
a penis erector including a penis-receiving body adapted for enabling the penis to be disposed within an interior space thereof, wherein the penis erector is adapted for enabling a negative pressure environment to be generated within the interior space of the penis-receiving body to cause the penis disposed within the penis-receiving body and exposed to the negative pressure environment to become erect; and
a containment body having a central passage extending between a first opening at a first end portion of the containment body and a second opening at a second end portion of the containment body, wherein a distal end portion of the penis erector is disposed within the central passage through the first opening, wherein the first end portion of the containment body is engaged with a mating portion of the penis erector to create a reflection chamber between the penis erector and the containment body, wherein the second end portion of the containment body is adapted for engaging a mating portion of an emitter body of an acoustic shock wave source for enabling acoustic shock waves to be emitted into the reflection chamber through the second opening from the acoustic shock wave source, and wherein material characteristics of the containment body cause the acoustic shock waves that impinge upon a surface of the containment body defining the central passage of the containment body to be reflected therefrom.

12. The device of claim 11, wherein the first end portion has a smaller cross-sectional area than a cross-sectional area of a central portion of the containment body.

13. The device of claim 11, wherein the first end portion of the containment body being engaged with the mating portion of the penis erector includes the first end portion of the containment body being in a sealed engagement with the mating portion of the penis erector.

14. The device of claim 11, wherein:
the penis erector includes a sealing body for providing a fluid-tight seal between the penis-receiving body and a body of the patient; and
the first end portion of the containment body being engaged with the mating portion of the penis erector includes the first end portion of the containment body being engaged with the sealing body.

15. The device of claim 11, wherein:
the penis erector includes a sealing body for providing a fluid-tight seal between the penis-receiving body and a body of the patient; and
the first end portion of the containment body being engaged with the mating portion of the penis erector includes the first end portion of the containment body being engaged with the penis erector at a location adjacent to an interface between the penis-receiving body and the sealing body.

16. The device of claim 15, wherein the first end portion of the containment body being engaged with the mating portion of the penis erector includes the first end portion of the containment body being in a sealed engagement with the mating portion of the penis erector.

17. The device of claim 11, wherein a centerline longitudinal axis of the containment body extends colinearly with a centerline longitudinal axis of the penis-receiving body.

18. A system for treatment of a penis of a patient, comprising:
a main body having a central passage extending between a first opening at a first end portion of the main body and a second opening at a second end portion of the main body, wherein material characteristics of at least a portion of the main body cause reflection of acoustic shock waves that impinge upon the at least a portion of the main body;
a first sealing body attached to the first end portion of the main body, wherein the first sealing body has a central passage for enabling at least a distal end portion of the penis to be positioned within the central passage of the main body, and wherein the first sealing body has a fluid-tight seal with the main body and is adapted to form a fluid-tight seal with a body portion of the patient adjacent to the penis;
a second sealing body attached to the second end portion of the main body, wherein the second sealing body inhibits leakage of fluid through the second opening of the main body from within the central passage of the main body and wherein material characteristics of the second sealing body enable acoustic shock waves to propagate therethrough; and
an acoustic shock wave module attached to the second end portion of the main body, wherein the acoustic shock wave module includes an acoustic shock wave source located in front of the second opening of the main body.

19. The device of claim 18, wherein the first sealing body is located between the acoustic shock wave source and the main body.

20. The device of claim 18, wherein a centerline longitudinal axis of the main body extends colinearly with a shock wave emittance axis of the acoustic shock wave source.

21. The device of claim 18, wherein the second end portion of the main body has a larger cross-sectional area than a cross-sectional area of a central portion of the main body and a cross-sectional area of the first end portion of the main body.

22. The device of claim 18, wherein the cross-sectional area of the central portion of the main body is approximately the same as the cross-sectional area of the first end portion of the main body.

23. A method for treating a penis of a patient, comprising the steps of:
positioning the penis within a penis-receiving space of a penis treatment device;
causing the penis-receiving space to become at least partially filled with an energy wave propagating media; and
emitting acoustic shock waves into the penis-receiving space after causing the penis-receiving space to become at least partially filled with the energy wave propagating media whereby the penis within the penis-receiving body is subjected to the acoustic shock waves, wherein the penis-receiving space is at least partially encompassed by a layer of material having material characteristics causing the acoustic shock waves that impinge upon the layer of material to be reflected therefrom.

24. The method of claim 23, wherein said causing the penis-receiving space to become at least partially filled with the energy wave propagating media-includes causing the penis-receiving space to become at least partially filled with the energy wave propagating media in a manner whereby a negative pressure environment is generated within the penis-receiving space.

25. The method of claim 23, wherein said causing the penis-receiving space to become at least partially filled with the energy wave propagating media includes causing a volume of air within the penis-receiving space to be reduced.

26. The method of claim 23, wherein the energy wave propagating media is liquid.

27. The method of claim 23, wherein said emitting acoustic shock waves into the penis-receiving space includes emitting the acoustic shock waves whereby a shock wave emittance axis of the acoustic shock waves extends colinearly with a centerline longitudinal axis of the penis-receiving space.

28. The method of claim 27, wherein said causing the penis-receiving space to become at least partially filled with the energy wave propagating media includes causing the penis-receiving space to become at least partially filled with the energy wave propagating media in a manner whereby a negative pressure environment is generated within the penis-receiving space.

29. The method of claim 23, wherein:
the penis-receiving space is defined by a penis-receiving body of a penis erector;
at least a portion of the penis-receiving body is within a central passage of a containment body; and
said emitting acoustic shock waves into the penis-receiving space includes emitting the acoustic shock waves into a reflection chamber jointly defined by the penis-receiving body and the containment body.

30. The method of claim 29, wherein said causing the penis-receiving space to become at least partially filled with the energy wave propagating media includes causing the penis-receiving space to become at least partially filled with water.
